(19) United States
(12) Patent Application Publication
(10) Pub. No.: US 2021/0299305 A1
(43) Pub. Date: Sep. 30, 2021

(54) Apparatus for cleaning an object

(71) Applicants: Samuel Frederick Cron, Philadelphia, PA (US); Christopher Melillo, Hillsborough, PA (US)

(72) Inventors: Samuel Frederick Cron, Philadelphia, PA (US); Christopher Melillo, Hillsborough, PA (US)

(21) Appl. No.: 17/301,158

(22) Filed: Mar. 26, 2021

Related U.S. Application Data
(60) Provisional application No. 63/002,075, filed on Mar. 30, 2020.

(51) Int. Cl. A61L 2/20; A61L 2/24

(52) U.S. Cl. CPC A61L 2/20; A61L 2101/20; A61L 2/24

(57) ABSTRACT

An apparatus that disinfects hand-held devices has a sealed interior disinfecting chamber designed to fit handheld devices of various sizes and surround them with disinfectant in a gaseous state. The base of the interior chamber has vertically disposed pegs of equal height that serve as an aerated pedestal on which to place a handheld device to allow gas to circulate beneath and around it. A fan evaporates disinfectant from the sealed chamber prior to unsealing the chamber.

### TECHNICAL FIELD

The present disclosure relates to apparatuses that disinfect hand-held objects and portable electronic devices. Relevant CPCs might include A61L2/24, Apparatus using programmed or automatic operation.

### BACKGROUND

Hand-held electronic devices are constantly in use and rarely cleaned. As a result, they collect dirt and pathogens from various environments, which get passed from device to hands and on to other surfaces.

Most current-market cell-phone disinfecting devices employ UV radiation. UV radiation has been found to degrade plastics by causing photochemical effects to the polymer structures of plastics. Absorbed UV energy can excite photons in plastics to release free radicals. Pure plastics cannot absorb UV radiation, but trace amounts of common impurities in plastics act as free-radical receptors.

In a 2020 report, the U.S. FDA stated that products generating UV light must shield a user from exposure. Failing to do so poses a potential health hazard depending on the wavelength, intensity, and exposure time of the UV radiation. In addition, UV radiation will not penetrate shadowed areas or areas unreachable by the path of radiation. This may result in inadequate disinfection. A University of New Hampshire study has found that most home UV light disinfection devices do not achieve the levels of germ-killing claimed in their advertisements.

A peristaltic pump moves the fluid through positive displacement. Fluid is held in a flexible tube inside a circular pump housing. Rolling parts (“rollers” or “wipers”) press on the flexible tube as they roll past it, forcing movement of the fluid. As the tube re-expands with fluid, it draws fluid back into it in the process of peristalsis.

Vaporized disinfectant has been commonly used to clean surfaces and is known in the art. In the context of this disclosure, a disinfecting vapor may be produced by mechanical, pneumatic, or electrical method.

Mechanically produced vapor is made by hand-driven, soft-mist asthma inhalers: by means of user-operated spring-loaded compression, the inhaler pressurizes fluid into mist.

Pneumatically produced vapor is made with jet nebulizers (also referred to as atomizers) which are commonly used in nebulizer inhalers. These devices force compressed air at high velocity through a liquid, resulting in an aerosol.

Electrical means may be of the ultrasonic or vibrating-mesh type. Ultrasonic wave nebulizers use an electronic oscillator to generate a high-frequency ultrasonic wave that vibrates a piezoelectric element. The vibrating element moves against a liquid reservoir to render the liquid into vapor. In vibrating-mesh technology, fluid is pushed through a vibrating mesh membrane to produce a vapor.

In the context of this embodiment, disinfectant fluid may be any combination of fluid with bactericidal and/or virucidal properties.

### SUMMARY

following presents a general summary of exemplary embodiments in order to provide a basic understanding of the aspects of the systems and methods disclosed herein without intending to identify key or critical elements or to delineate the scope of the present disclosure.

In accordance with example embodiments of the present disclosure, an apparatus and method for disinfecting portable electronic devices has a sealed interior disinfecting chamber designed to fit portable electronic devices of various sizes is disclosed. A fluid handling system that delivers a fluid into the disinfecting chamber, thus surrounding a portable electronic device with disinfecting fluid is further disclosed. A drying system that removes disinfecting fluid from the chamber is again further disclosed.

A first iteration uses electronic components to control a disinfection process. The apparatus includes a disinfecting chamber with a disinfecting-fluid reservoir; a sealing lid with supporting members that hold the portable electronic device while allowing vapor to flow over all sides of the device; a second reservoir for waste fluid; a pump and plumbing; and power and controlling electronics. In operation, a pump moves fluid from the disinfectant reservoir to a compressor and on to vapor generators, which emit vapor into a chamber where a portable electronic device (or similar small device) has been placed. After a measured disinfecting interval, a fan evacuates the disinfecting chamber, condensing the disinfecting vapor into waste fluid, which is directed to a drain that has an activated-charcoal cleaning filter. The apparatus includes an audio-visual display that plays music, illustrates the disinfecting process, and may show advertising messages.

In some embodiments, the base of the interior chamber has vertically disposed supports, such as pegs of equal height, which serve as an aerating pedestal on which to place a handheld device to allow gas and air to circulate beneath and around it. In other embodiments, a mesh, screen or horizontal linear structural member suspends objects in the chamber. A fan may distribute and evaporate sprayed disinfectant throughout the sealed chamber. Plumbing from the disinfecting fluid leads to a spray port in the sealed chamber. Pressurized disinfectant fluid is held in a storage compartment.

A CPU runs software to turn components on or off. Electronics control the timing and function of one or more proximity sensors, as well as a proximity switch, spray mechanisms, fans and valves. At least one proximity sensor detects the presence or absence of an object in the disinfecting chamber. A proximity switch detects the opening or closing of the chamber lid. An actuated valve controls the spraying of disinfectant into the chamber. Software couples a signal from the proximity switch with a signal from a proximity sensor. If the lid is opened and closed, and a device is detected by a proximity sensor, a disinfectant sequence commences. If the lid is opened then closed and a device is not detected by a proximity sensor, no action is taken and the apparatus remains off.

In use, an object such as a handheld electronic device is placed in the disinfectant chamber and a user closes the lid. The closing action toggles the proximity switch to “on,” which signals to the control electronics that the lid has been closed. Control electronics measure output from a proximity sensor, which measures distance from its position to the nearest surface. A detected distance between proximity sensor and placed object initiates a disinfecting sequence.

In some embodiments, the steps of a disinfecting sequence is displayed on an audio-visual screen as they occur. In other embodiments, LEDs of varying colors flash to indicate steps of the sequence as they occur. A sequence begins when a lid to a disinfecting chamber is closed, it is then locked and subsequently the sequence continues by powering on an actuated valve that sprays disinfectant through the spray port into the disinfecting chamber.

The control electronics opens a valve wherein a given volume/minute of disinfectant is sprayed into the chamber. A timer in the control electronics determines a duration during which a volume-per-minute is sprayed, effectively dispensing a measured quantity of disinfectant. After disinfection, the timer signals the power electronics to close the actuated valve. Once the valve is closed, control electronics actuate a fan to evacuate the chamber of disinfectant and dry the handheld device. In a preferred embodiment, an audio-visual display indicates that drying is complete, in other embodiments, an LED may indicate that drying is complete; or an unlocking mechanism may be activated so that the chamber may be opened to retrieve the sanitized device. When evacuation is complete, the disinfected electronic device may be removed by lifting the apparatus's lid and removing the electronic device.

In some embodiments, once the lid is re-closed, a proximity switch signals the power electronics to turn on a proximity sensor to measure distance from its position to the nearest surface, in this case the lid. Indication of the closed lid powers off the apparatus.

One skilled in the art understands that the term fluid may refer to a liquid or a gas. Other objects and features will become apparent from the following detailed description considered in conjunction with the accompanying drawings. It is to be understood, however, that the drawings are designed as an illustration and not as a definition of the limits of the invention.

### BRIEF DESCRIPTION OF THE DRAWINGS

FIG. 1 is a front perspective view of an example embodiment;

FIG. 2 is a front perspective view of the example embodiment of FIG. 1 with outer housing removed;

FIG. 3 is a rear perspective view of the example embodiment of FIG. 1 with outer housing removed;

FIG. 4 is a bottom, rear, perspective view of the example embodiment of FIG. 1 with outer housing removed;

FIG. 5 is a detail view of a disinfecting chamber of the example embodiment of FIG. 1;

FIG. 6 is a detail view of a disinfecting chamber of the example embodiment of FIG. 1 with an electronic device placed inside;

FIG. 7 is a bottom perspective view of an example of a door of the example embodiment of FIG. 1;

FIG. 8 is a front perspective view of an iteration of the example embodiment;

FIG. 9 is a perspective, partial section view of the embodiment of FIG. 7.

FIG. 10 is a diagram of a method of using the example apparatus.

### DESCRIPTION

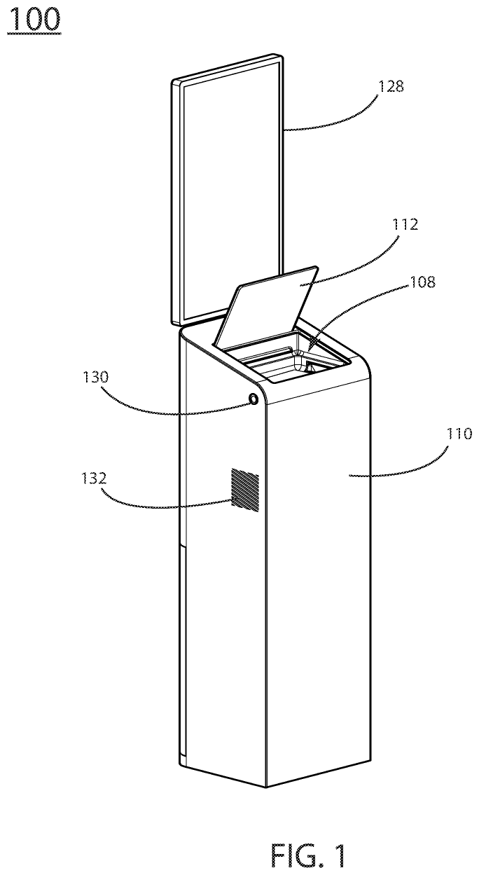

In FIG. 1, an example embodiment of an apparatus for disinfecting a handheld object has a housing 110 around internal components, and a lid 112 that covers a disinfecting chamber 108. An emergency-stop switch 130 is accessible to one in proximity of the disinfecting chamber 108. An audio-visual screen 128 is disposed near the housing 110. One skilled in the art understands that an audio-visual screen may display advertising messages along with sound and may also illustrate or describe the sanitization process that is carried out in the disinfecting chamber 108.

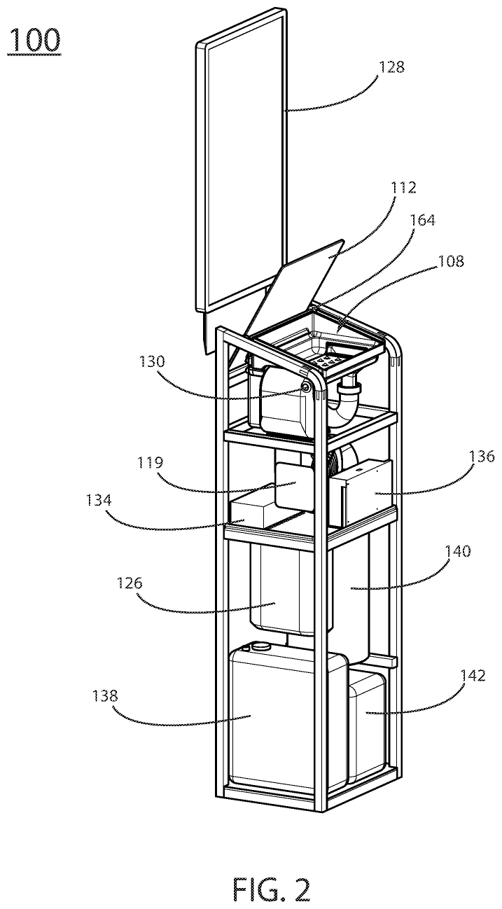

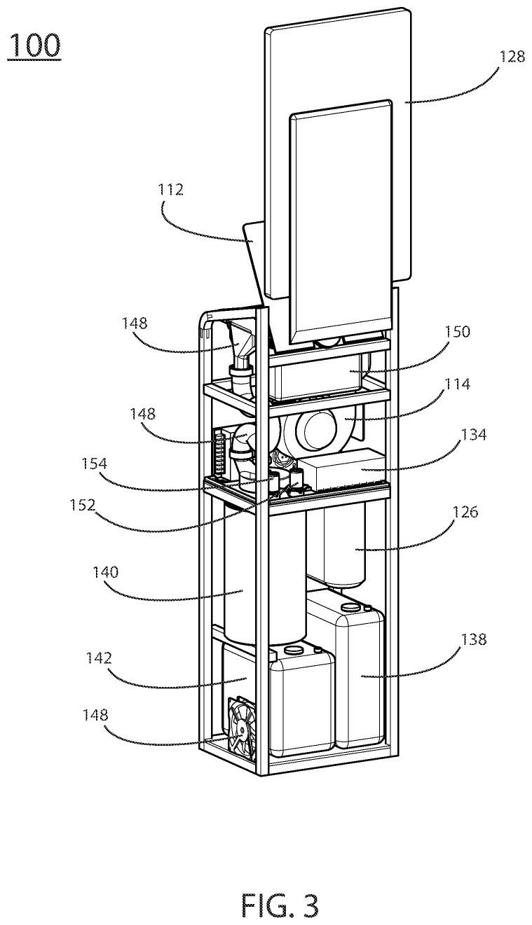

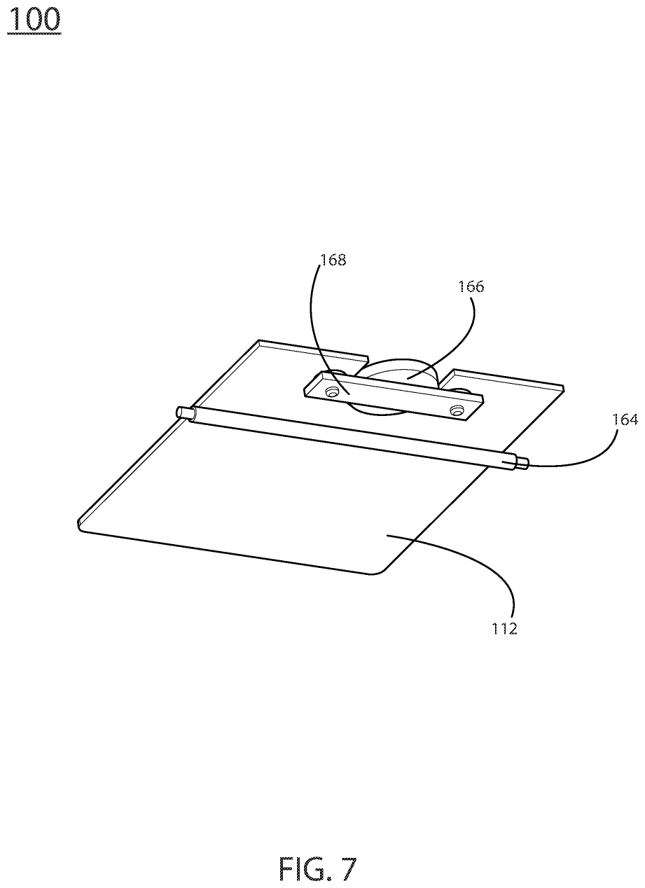

In FIGS. 2, 3 and 4, the apparatus of FIG. 1 is shown with the outer housing 110 removed. A lid 112 is configured to close over a disinfecting chamber 108. The lid 112 pivots on a continuous hinge 164 and is weighted to remain normally open. (See FIG. 7 for a detailed description of the lid-and-closure mechanism). An audio-visual screen 128 is proximal to the disinfecting chamber 108 such that one standing near the apparatus may view and hear what is displayed. One skilled in the art understands that advertisements, entertainment and a narration or description of the disinfecting process may be displayed on the audio-visual screen 128.

The disinfecting chamber 108 is supported by components that provide features and functions for surrounding an electronic device with a disinfectant vapor in a closed environment for a sufficient period of time to kill bacteria and viruses on the electronic device and then to remove the disinfectant vapor and dry the electronic device prior to removal. In normal operation, the door remains locked in a closed position for the duration of the procedure. An emergency-stop switch 130 allows one to stop the process and remove the electronic device at any time.

A reservoir 138 is configured to hold disinfectant solution. A second reservoir 142 is configured to hold waste fluid. One skilled in the art understands that components are plumbed in communication with one another, and although all plumbed lines may not be visible, their function is understood. A compressor 126 sends compressed air to vapor generators 156 which are housed in a vapor-generator housing 150. A pump 152 moves fluid from the disinfectant reservoir 138 to vapor generators 156. In some embodiments the pumps 152 and 154 are peristaltic pumps. In some embodiments, a vapor is generated by vapor generators 156. Vapor may be produced by a Venturi-impaction nebulizer or by other means of aerosol production, such as ultrasonic diaphragms, perforated piezoelectric diaphragms, spray nozzles, air-assisted nozzles or electrostatic sprayers. In some embodiments, a vapor may be electrically charged to promote droplet dispersion and adhesion.

After an electronic device has been surrounded by vapor for a measured duration, an on/off controller signals a fan 114 to fill the disinfectant chamber 108 with ambient air that moves fluid droplets and vapor to a drain 148 while drying the interior of the chamber and items within. In some embodiments, the drain 148 has a toroidal pathway that causes vapor to condense. The drain is in fluid communication with a charcoal filter 140 that in turn is in fluid communication with a waste reservoir 142. An enclosure fan 148 pulls air through a vent 132 and adjacent filter 119 so as to move air through the enclosure to keep internal components dry.

A power supply 136 supplies power to control electronics in electronics housing 134. Control electronics supply power to the electronic components in an order that carries out the process of disinfecting a portable electronic device. A plenum 127 creates a vapor barrier between power electronics in the upper region of the apparatus and fluid storage components in the lower portion of the apparatus.

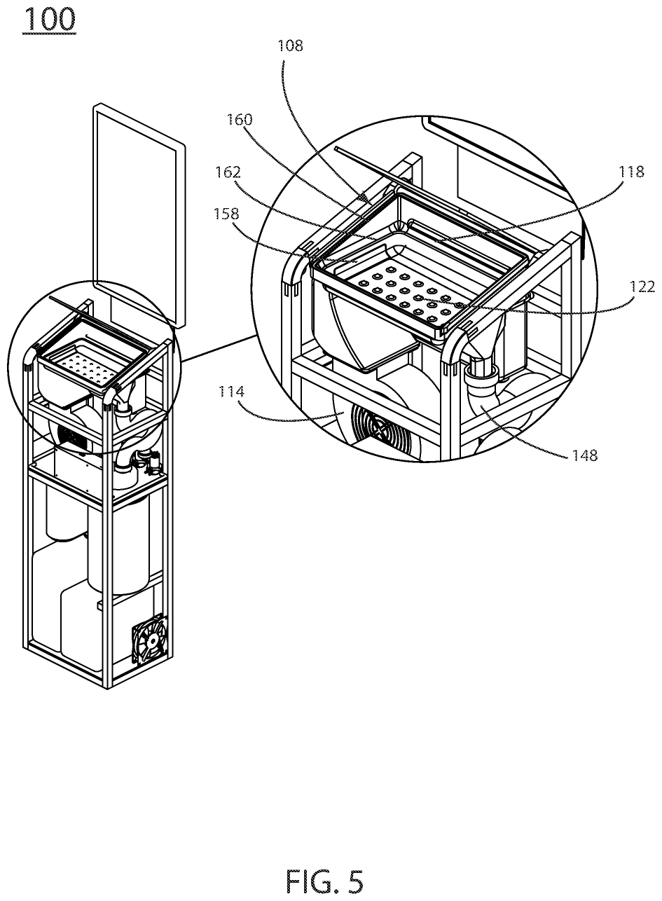

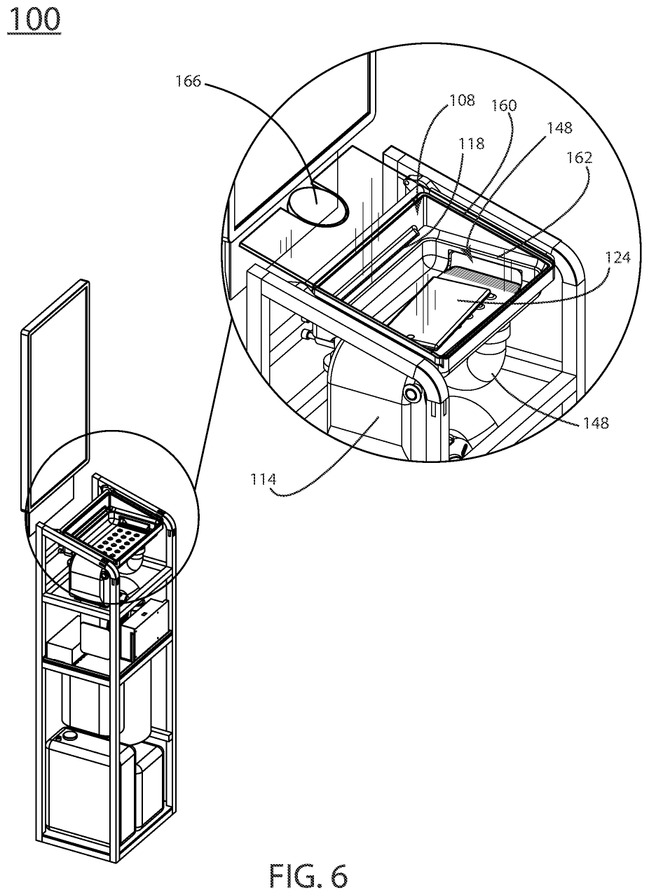

Referring to FIGS. 5 and 6, the disinfecting chamber 108 is shown with and without a portable electronic device 124 placed in it. A portable electronic device 124 may be placed on an array of supporting members 122 that hold the portable electronic device 124 to enable flow of vapor over all sides of the device. Lights 162 illuminate the interior of the disinfecting chamber 108. A gasket 160 seals against the lid 112 when it is closed. FIG. 6 shows a transparent lid 112 in a closed position. One skilled in the art understands how a magnet 166 may engage with an electromagnet in the housing 110 (FIG. 1) to keep the lid 112 closed during a disinfecting process, and that the electromagnet may release the magnet 166 when the process is complete.

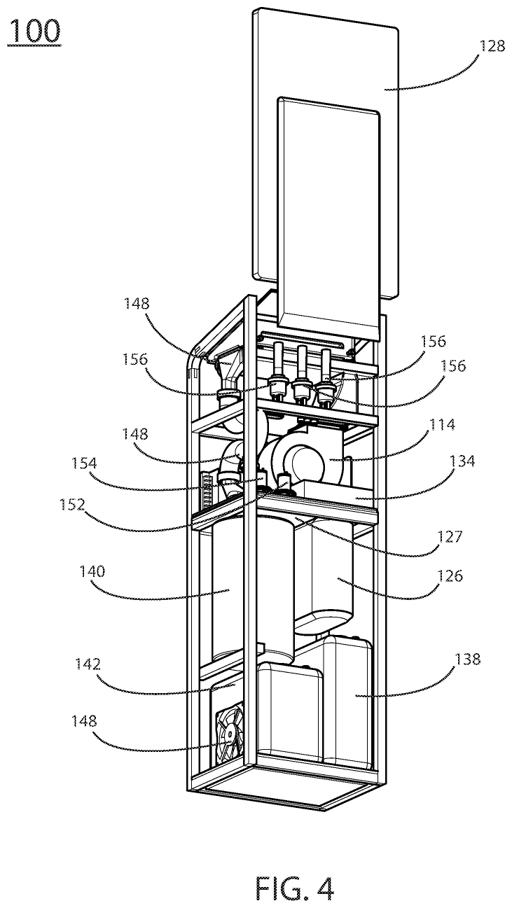

Vapor generated by vapor generators 156 (FIG. 4) enters the disinfecting chamber 108 through vapor port 118. The vapor may surround the portable electronic device 124 that is placed atop the supporting members 122. When closed, a damper 158 prevents the flow of vapor out of the chamber 108 and into the ductwork leading to the fan 114. When the disinfecting process is complete, the damper 158 opens and air from the fan 114 is forced past the damper 158 to evacuate the chamber as the air flows past the portable electronic device 124 toward the drain 148. Fluid droplets and vapor flow into the drain 148 where the vapor is condensed. The resulting fluid flows into the charcoal filter 140 and waste reservoir 142 (FIG. 4).

In an example disinfecting process, a portable electronic device is placed in the disinfecting chamber 108, and the door 112 is closed against the seal 160 to initiate the process.

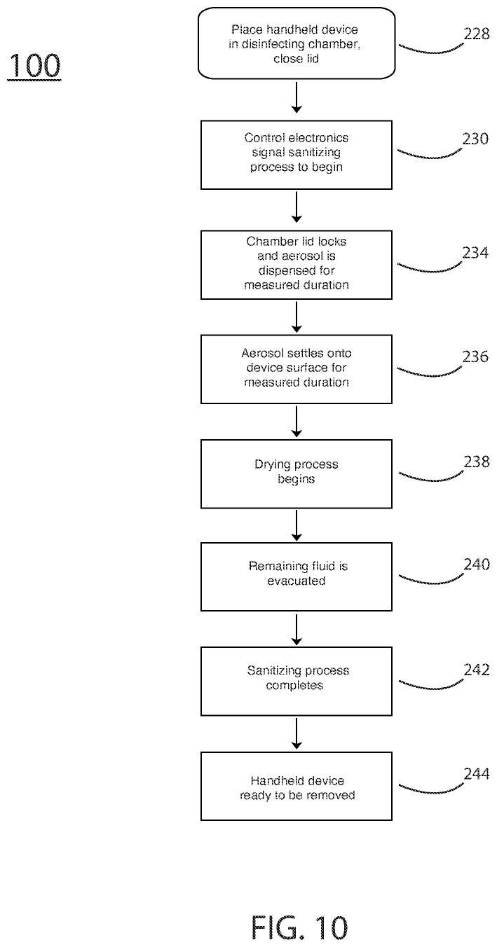

In the bottom-perspective view of FIG. 7, an example lid 112 is illustrated. The lid 112 pivots on a continuous hinge 164 that is pivotally mounted to the apparatus proximal to the chamber 108 so that it closes the opening of the chamber 108. The lid 112 has a weight 168 affixed to the rear of the door such that the door pivots on the continuous hinge 164 and remains in a normally open position. A magnet 166 is affixed to the rear of the door so that when the door is pivoted to a closed position, the magnet 166 joins an electromagnet to hold the door closed during the disinfecting procedure (FIG. 10). One skilled in the art is familiar with electromagnetic locking mechanisms.

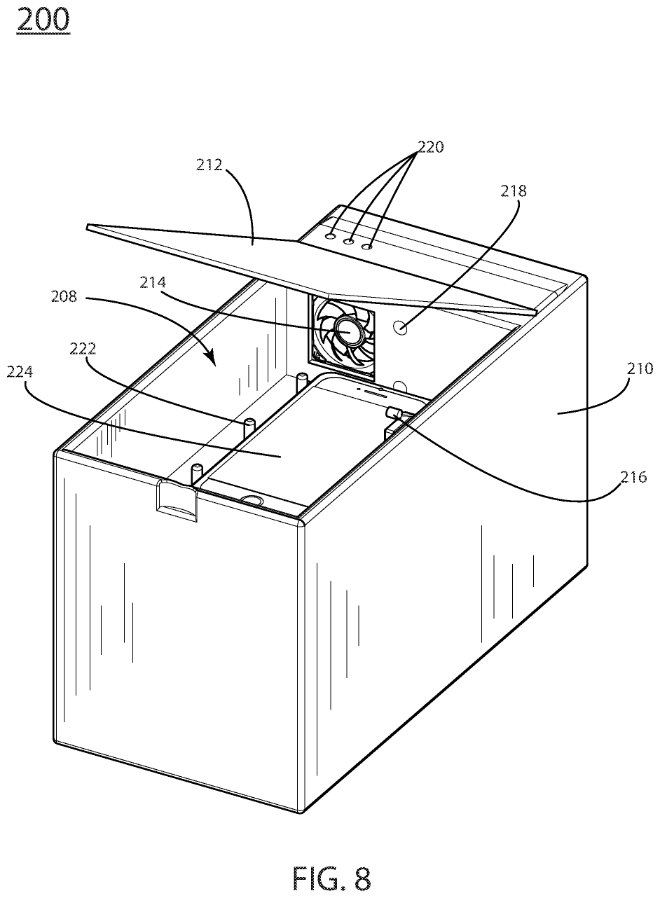

In FIG. 8, a housing 210 includes a lid 212 and a proximity switch 216. The lid 212 covers a disinfecting chamber 208. The proximity switch is tripped when the lid 212 is closed. Supporting members 222 hold an electronic device 224 in a position to enable airflow on all sides of the electronic device 224. A spray port 218 directs sprayed disinfectant into the chamber 208. A fan 214 evaporates disinfectant that has been sprayed into the chamber. In some embodiments the disinfectant is in a gaseous state.

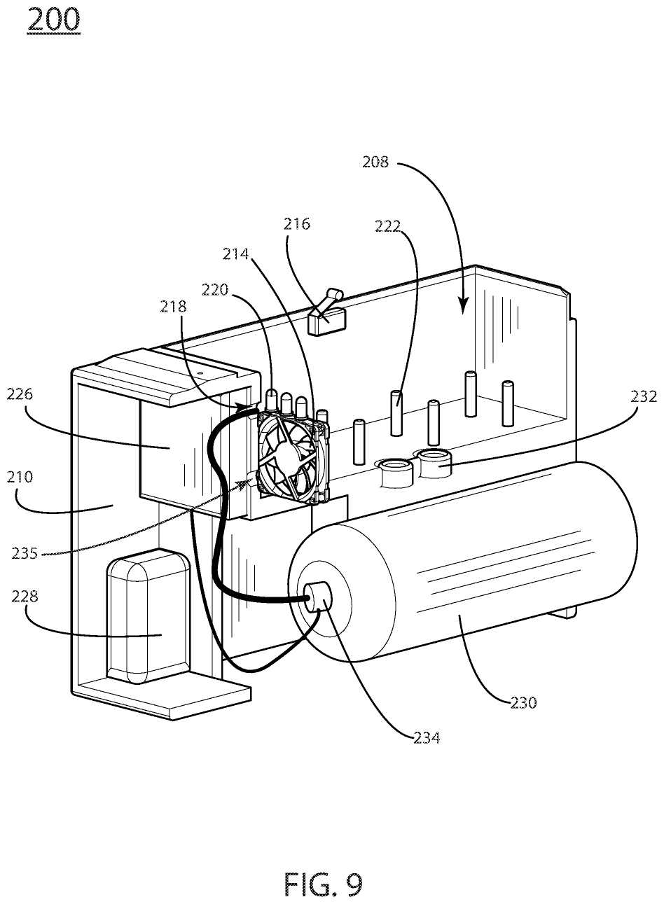

In FIG. 9, electronics control the timing and function of at least one sensor, at least one spray mechanism and at least one fan. An electronic device is placed in the disinfecting chamber 208 and sprayed with disinfectant. Disinfectant resides in a pressurized container 231. Disinfectant release is controlled by an actuated valve 235 that permits the flow of gaseous disinfectant through plumbing that directs the disinfectant to a spray port 218. In an example embodiment, at least one LED 220, an actuated valve 235, at least one proximity sensor 232, proximity switch 216 and fan 214 are electronically coupled with the control electronics 227. A power source 229 provides power to the electronic components. One skilled in the art understands that some disinfectants need not be evacuated from a chamber prior to opening the chamber and in such example embodiments a fan would not be needed.

When the lid (FIG. 8) is closed, it toggles the proximity switch 216 to “on.” The proximity switch 216 signals the control electronics that the lid has been closed. The control electronics measure the output from at least one proximity sensor 232, which measures the distance from its position to the nearest surface. If a proximity sensor 232 measures the distance from its position to the lid, the control electronics 227 do not initiate a sequence and the system remains powered off. In some embodiments a pair of sensors measures the distance as one emits a sound wave and the other sensor of the pair receives the returning sound wave. One skilled in the art understands that various methods exist for measuring proximity.

If a proximity sensor 232 measures the distance from itself to an item placed on supporting members 222, the control electronics initiate a sequence. The sequence flashes control LEDs to signal the steps of the sequence as they occur. In some embodiments three LEDs 220 signify a power-on step, an item-recognized-by-proximity-sensor step 232, and a sequence-initiated step. One skilled in the art understands that various LEDs may be used in various combinations. The sequence begins by powering the actuated valve 235 to spray a measured amount of disinfectant through the spray port 218 and into the disinfecting chamber 208. A timer in the control electronics determines an amount of and timing of disinfectant sprayed. The timer then signals the power electronics to close the actuated valve 235. Once the actuated valve 235 is closed, the control electronics power a fan 214 to evacuate the disinfecting chamber 208 of disinfectant, completing the process. The provided electronic device 224 (FIG. 8) may be removed by manually lifting the lid 212 (FIG. 8) and removing the electronic device 224 (FIG. 8). Upon closing the lid 212 (FIG. 8) the proximity switch 216 signals the power electronics 227, which turns on a proximity sensor 232, which measures distance from its position to the nearest surface, in this case the lid 212 (FIG. 8), indicating the absence of a placed handheld electronic device. Having detected no handheld electronic device, the apparatus receives a message from the power electronics 227 to turn off.

FIG. 10 is a flowchart illustrating the disinfectant process. A handheld device is placed in the disinfecting chamber and lid is closed 228. Control electronics signal sanitizing process to begin 230. Chamber lid locks and aerosol is dispensed for measured duration 234. Sanitizing aerosol settles onto device surface for measured duration 236. Drying process begins 238. Remaining fluid is evacuated 240. Video and/or audio signals completion of sanitization 242. Handheld device is removed from chamber 244.

The example embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the Invention.

1. An apparatus for sterilizing a portable electronic device comprising:
a chamber having a normally open cover; and
a locking device configured to hold said normally open cover in a closed position; and
a platform with protrusions configured to hold a portable electronic device while allowing for maximum exposure of outer surfaces of said portable electronic device; and
at least one nebulizer; and
a vessel for storing fluid sanitizer; and
a first inlet in said chamber and in fluid communication with said at least one nebulizer for delivery of vapor from said nebulizer through said first inlet, into said chamber; and
a second inlet in said chamber and in fluid communication with a fan for moving ambient air through said second inlet, into said chamber; and
a drain in fluid communication with said chamber; wherein
a portable electronic device is placed in said chamber on said protrusions, the normally open cover is closed, a vaporized sanitizing fluid formed by said at least one nebulizer is introduced to said chamber through said first inlet, filling said chamber with sanitizing vapor, said fan moves air into said chamber through said second inlet, condensing said vapor, and moving said vapor to said drain for removing said vapor and drying said portable electronic device.

2. The apparatus of claim 1 further comprising an audio-visual screen fixedly engaged with the apparatus.

3. The apparatus of claim 1 further comprising a lock for closing said normally open cover.

4. The apparatus of claim 1 further comprising a charcoal filter in fluid communication with said drain.

5. The apparatus of claim 1 wherein the fluid sanitizer is a solution of benzenesulfonic acid.

6. The apparatus of claim 1 wherein the fluid sanitizer is a solution of benzenesulfonic acid and L-Lactic acid.

7. The apparatus of claim 1 wherein the fluid sanitizer is solution of 2-dodecylbenzenesulfonic acid.

8. The apparatus of claim 1 wherein the fluid sanitizer is a solution of 2-dodecylbenzenesulfonic acid and L-Lactic acid.

9. The apparatus of claim 1 wherein said drain is in fluid communication with a waste fluid storage vessel.

10. The apparatus of claim 1 further comprising
an electro-magnetic latch on said normally open cover; and
control circuitry; and
the control circuitry programmed to:
read a signal from the electro-magnetic latch on said normally open cover when said cover is closed; and
maintain said electro-magnetic latch in a closed position; and
send power to said at least one nebulizer; and
wait a first period of time; and
send power to said fan; and
wait a second period of time; and
release said electro-magnetic latch allowing said normally open cover to open;
wherein
said control circuitry controls the operation of sanitizing and drying an object commencing with the closing of said normally open cover.

11. An apparatus for sterilizing a portable electronic device comprising:
a chamber having a normally open cover; and
an electro-magnetic latch on said normally open cover configured to hold said normally open cover in a closed position once closed; and
platform with protrusions configured to hold a portable electronic device while allowing for maximum exposure of outer surfaces of said portable electronic device; and
a compressed gas source; and
at least one nebulizer; and
a vessel for storing fluid sanitizer; and
a pump in fluid communication with said vessel for storing fluid sanitizer; and
a first inlet in said chamber and in fluid communication with said at least one nebulizer for delivery of vapor from said nebulizer through said first inlet, into said chamber; and
a second inlet in said chamber and in fluid communication with a fan for moving ambient air through said second inlet, into said chamber; and
an electro-mechanical damper movably engaged with said second inlet; and
a drain in fluid communication with said chamber; and
a charcoal filter in fluid communication with said drain; and
a controller configured to perform a controlled process by the control of the flow of power to said electro-magnetic latch, said at least one nebulizer, said damper and said fan; wherein
a portable electronic device is placed in said chamber on said protrusions and said normally open cover is closed, said controller sends power to said electro-magnetic latch to hold said normally open cover in a closed position, said controller sends power to said electro-mechanical damper, closing said electro-mechanical damper, said controller sends power to said pump, said fluid sanitizer is pumped by said pump to said at least one nebulizer, said controller controls the flow of compressed gas to said at least one nebulizer, sanitizing fluid vaporized by said at least one nebulizer is introduced to said chamber through said first inlet, filling said chamber with sanitizing vapor, said controller sends power to said electro-mechanical damper, opening said electro-mechanical damper; said controller sends power to said fan which moves air through said second inlet, into said chamber for drying said portable electronic device and condensing said vapor and moving said vapor toward said drain for removing said vapor and fluid from said chamber, and for drying said portable electronic device; and said controller sends power to said electro-magnetic latch to open said electro-magnetic latch to open said normally open cover for removal of said portable electronic device.

12. The apparatus of claim 11 further comprising a touch screen for displaying information about the process controlled by said controller and for displaying audio-visual advertising.

13. The apparatus of claim 11 further comprising a switch configured to signal said controller to stop said controlled process and open said electro-magnetic latch.

14. The apparatus of claim 11 further comprising a waste fluid container in fluid communication with said charcoal filter; wherein waste fluid drained from said chamber is filtered through said charcoal filter and moved to said waste fluid container for removal.

15. The apparatus of claim 11 wherein the fluid sanitizer is a solution of benzenesulfonic acid.

16. The apparatus of claim 11 wherein the fluid sanitizer is a solution of benzenesulfonic acid and L-Lactic acid

17. The apparatus of claim 11 wherein the fluid sanitizer is a solution of 2-dodecylbenzenesulfonic acid.

18. The apparatus of claim 117 wherein the fluid sanitizer is a solution of 2-dodecylbenzenesulfonic acid and L-Lactic acid.

19. The apparatus of claim 11 wherein said drain is in fluid communication with a waste fluid storage vessel.

20. A method for sanitizing a portable electronic device employing the apparatus of claim 11, the method comprising:
placing a portable electronic device in said chamber; and
initializing a sanitizing process; and
locking said normally open cover; and
vaporizing a sanitizing fluid; and
dispensing said sanitizing fluid in vapor form at a constant rate for a measured duration; and
initializing a drying process by powering said fan; and
evacuating remaining fluid from said chamber; and
signaling completion of said sanitizing process; wherein
said portable electronic device is removed from said chamber.
